(19) United States
(12) Patent Application Publication
(10) Pub. No.: US 2007/0092907 A1
(43) Pub. Date: Apr. 26, 2007

(54) Methods of screening compound probes

(76) Inventors: Sanjiv S. Gambhir, Portolla Valley, CA (US); Zhen Cheng, Mountain View, CA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/584,191

(22) Filed: Oct. 20, 2006

Related U.S. Application Data
(60) Provisional application No. 60/729,255, filed on Oct. 21, 2005.

(51) Int. Cl. C40B 30/06; G01N 33/53

(52) U.S. Cl. 435/6; 435/7.1; 436/86

(57) ABSTRACT

Briefly described, embodiments of this disclosure include methods for identifying compound probe candidates, methods of screening compound probe candidates, methods of preparing molecular imaging probes, high throughput methods for identifying molecular imaging probes in a library, and the like.

### CROSS-REFERENCE TO RELATED APPLICATION

This application claims priority to U.S. provisional applications entitled, “Matrix Assisted Laser Desorption Ionization (MALDI) Support Structures and Methods of Making MALDI Support Structures,” having Ser. No. 60/729,255, filed on Oct. 21, 2005, which is entirely incorporated herein by reference.

### BACKGROUND

Molecular imaging is a fast growing research discipline that provides the ability to study diseases non-invasively in living subjects at the molecular level. Advances in molecular and cell biology have generated many powerful tools for rapid identification and validation of new targets. When compared to the current approximation of 500 molecular targets, it is estimated that 5,000-10,000 drug targets will be discovered and explored in the near future. Therefore, in order to fully realize the power of molecular imaging, it is highly desirable to be able to develop numerous probes in a relatively short period of time and use them to image specific molecular targets.

Technologies such as combinatorial techniques, computer aided drug design, and high-throughput drug screening have been employed for therapeutic drug development, and these techniques have pushed drug discovery forward dramatically. However, to date, the strategies for developing imaging probes, especially radiolabeled tracers, remain essentially the same.

The general procedure typically starts with the identification of lead biological compounds or structure components, incorporation of radionucleotides into the lead compound and/or its derivatives, followed by evaluation of radiolabeled agents in cell culture and in animals, including humans. This strategy requires the preparation of radioactive probes before any biological activity evaluation, which can be very time consuming and expensive. Moreover, the method of incorporating the radioisotope into the lead compound while preserving the probe's biological properties is not an exact science.

There are many criteria for a good molecular imaging probe. The ability of a probe to overcome biological delivery barriers such as the cell membrane is one of the key issues that may ultimately determine its potential utility in practice.

Therefore, the earlier this information can be obtained, the sooner the probe development strategy and any conclusions about its potential utility may be reached.

### SUMMARY

Briefly described, embodiments of this disclosure include methods for identifying compound probe candidates, methods of screening compound probe candidates, methods of preparing molecular imaging probes, high throughput methods for identifying molecular imaging probes in a library, and the like.

An embodiment of a method, among others, includes: providing a library of unlabeled compound probes, wherein each compound probe contains a first element, wherein the first element has at least one corresponding radioisotope; introducing each compound probe to a sample; incubating each compound probe with the sample; quantifying the amount of each compound accumulated by each sample using a mass spectrometry system; selecting one or more compound probes based on criteria; and

labeling each of the selected compound probes by replacing the first element with one of the corresponding radioisotopes.

An embodiment of a high throughput method for identifying molecular imaging probes in a library, among others, includes: providing a library of unlabeled compound probes, wherein each compound probe contains a first element, wherein the first element has at least one corresponding radioisotope; introducing each compound probe to a sample; incubating each compound probe with the sample; quantifying the amount of each compound accumulated by each sample using a matrix assisted laser desorption/ionization (MALDI) time-of-flight mass spectrometry system; and selecting one or more compound probes based on criteria.

An embodiment of a high throughput method for identifying molecular imaging probes in a library, among others, includes: providing a library of compound probes, wherein each compound probe contains a label selected from: a fluorophor, a MRI contrast agent, and a CT contrast agent; introducing each compound probe to a sample; incubating each compound probe with the sample; quantifying the amount of each compound accumulated by each sample using a mass spectrometry system; selecting one or more compound probes based on criteria.

### BRIEF DESCRIPTION OF THE DRAWINGS

Aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates the chemical structure and molecular mass (M.W.) of a library of phosphonium cations: (4-bromobutyl) triphenyl-phosphine bromide (Compound #1, BrTPP), butyltriphenylphosphonium chloride (Compound #2, BuTPP), (4-carboxybutyl)triphenyl-phosphonium bromide (Compound #3, CoTPP), methyltriphenyl phosphonium bromide (Compound #4, MeTPP), tetraphenylphosphonium bromide (Compound #5, TPP), triphenyl(2-pyridylmethyl) phosphonium chloride hydrochloride (Compound #6, PyTPP), tetrabutylphosphonium bromide (Compound #7, TBuP), 4-Fluorophenyltriphenyl phosphonium (Compound #8, FTPP), tetra-4-fluorophenylphosphonium iodide (Compound #9, F4TPP), and 4-fluorobenzyl-triphenylphosphonium iodide (Compound #10, FBnTP).

FIG. 2 illustrates representative MALDI-TOF-MS spectra for the time course cell uptake of TPP. Ion intensity of internal standard ([MeTPP]⁺ m/z 277.1) was set as 100 in each spectrum. Ion intensity of analyte, TPP ([TPP]⁺ m/z 339.1), increases over time and reaches a maximum uptake at 60 min incubation time. The peak area of the [M]⁺ ion was determined, and the ion intensity ratio (TPP/MeTPP) was calculated. Therefore, the amount of TPP in the cells at each time point can be quantified in conjunction with the calibration curve. (The unlabeled peaks are generated from the matrix).

FIG. 3 illustrates a representative MALDI-TOF-MS spectrum for cell uptake of TbuP (Compound #7) at 90 min incubation. The TBuP peak ([M]⁺: m/z 259.2) is relatively low but is still easily identified since the signal to noise ratio is over 5.

FIG. 4 illustrates the linear correlation of the MALDI-TOF-MS ion-intensity ratios [phosphonium cations (PCs)/MeTPP] versus the mole ratios (PCs/MeTPP), with 1.0 μM MeTPP as internal standard. 1 μL of solution or suspension containing 0.1-1.5 pmol of phosphonium cations was used for each analysis. Averages of results were from triplicate analyses. The lines are based on linear least-squares fitting of each of these sets of data points.

| | |
|---|---|
| TPP:MeTPP Y = 0.012225 + 1.00453X | r² = 0.99915 |
| BrTPP:MeTPP Y = 0.00815 + 0.40528X | r² = 0.99713 |
| BuTPP:MeTPP Y = 0.02725 + 1.66651X | r² = 0.98602 |
| PyTPP:MeTPP Y = 0.01386 + 0.89319X | r² = 0.99525 |
| TBuP:MeTPP Y = 0.01565 + 1.05212X | r² = 0.99872 |
| FTPP:MeTPP Y = 0.0215 + 1.06846X | r² = 0.99824 |
| F4TPP:MeTPP Y = 0.00584 + 0.70597X | r² = 0.99918 |
| FBnTP:MeTPP Y = 0.0176 + 0.83203X | r² = 0.991 |

FIG. 5 illustrates the C6 cell uptake of TPP over time at room temperature. The unlabeled TPP uptake was determined with MALDI-TOF-MS, and tritiated TPP uptake was measured with scintillation counting. Values are expressed as mean percentage of cell uptake±standard deviation (S.D.) of three independent determinations. The time course of TPP influx, as determined with the two techniques, exhibited almost identical patterns and the same levels of uptake with no significant difference (P<0.05).

FIG. 6 illustrates C6 cell uptake of various phosphonium cations over time at room temperature as determined by MALDI-TOF-MS. Values are expressed as mean percentage of cell uptake±S.D. of three independent determinations.

FIG. 7 illustrates the effects of various concentrations of protonphore CCCP, K⁺-ionophore valinomycin, and high K⁺ HEPES buffer on the uptake of 5 μM FTPP. Each value represents the mean of three independent experiments. Values are expressed as mean percentage of normalized uptake±S.D. of three independent experiments.

FIG. 8 illustrates the effects of various concentrations of protonphore CCCP, and high K⁺ HEPES buffer on the uptake of 5 μM F4TPP and FBnTP. Each value represents the mean of three independent experiments. Values are expressed as mean percentage of normalized uptake±S.D. of three independent experiments.

### DETAILED DESCRIPTION

Embodiments of the present disclosure will employ, unless otherwise indicated, techniques of mass spectrometry, synthetic organic chemistry, biochemistry, molecular biology and the like, that is within the skill of the art. Such techniques are explained fully in the literature.

The following examples are put forth so as to provide those of ordinary skill in the art with a complete disclosure and description of how to perform the methods and use the compositions disclosed and claimed herein. Efforts have been made to ensure accuracy with respect to numbers (e.g., amounts, temperature, etc.) but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range.

All patents, patent applications, and publications mentioned herein are hereby incorporated by reference in their entireties.

Discussion

Methods for identifying compound probe candidates, methods of screening compound probe candidates, and methods of preparing molecular imaging probes, are provided. In particular, embodiments of the present disclosure include high throughput methods for identifying and screening compound probe elements such as molecular imaging probes. It should be noted that the term “screening” refers to the identification of one or more compound probe candidates from among large collections of compound probe candidates, for example a library of unlabeled compound probe candidates.

In general, methods of the present disclosure use a mass spectrometry system (e.g., matrix assisted laser desorption/ionization (MALDI) time-of-flight mass spectrometry system) to rapidly analyze, in a high throughput manner, a library of unlabeled compound probes (e.g., molecular imaging probe candidates) to identify and select unlabeled compound probes of the library to be labeled and analyzed for use as molecular imaging probes. The unlabeled compound probes include compounds having one or more elements that have a non-radioactive isotope, which can subsequently be switched to the radioactive isotope of the element (e.g., F-19 (F-18), C-12 (C-11), I-127 (I-125, I-124, I-131, I-123), and the like) after the library of compound probes is screened. In another embodiment, the compound probe can include a labeling moiety such as, but not limited to, a fluorophor, a magnetic resonance imaging (MRI) contrast agent, or a computer tomography (CT) contrast agent.

The methods of the present disclosure can be used to rapidly and inexpensively identify and/or screen a library of unlabeled compound probes for select compound probes (e.g., those satisfying certain criteria) to be further investigated and evaluated (e.g., by replacing the element with a radioactive isotope of the element) as molecular imaging probes. In some instances, these methods remove the expensive and time-consuming steps of labeling each of the compound probes in a library with a label and then evaluating each labeled compound probe. In addition, these methods provide a robust method to identify and/or screen a library of compound probes without the interference of a label, which, in some instances, can interfere with the compound probe's biological properties.

For example, in embodiments using prospective PET or SPECT agents, the library of unlabeled compound probes contain the non-radioactive isotopes, which can be subsequently changed to the radioactive isotopes after the screening process is complete. In another example, the labeling moiety may be a fluorophor, a MRI contrast agent, or a CT contrast agent that is already present in the compound probes.

In general, each compound probe of the library is introduced to a sample and incubated with the sample. The sample can include, but is not limited to, a living host, a cell culture, a tissue, an organ, a tumor, an organelle, an array of bound target molecules, combinations thereof, and the like. The conditions for incubation are dependent, at least in part, upon the sample type, the compound probes, conditions of the sample, time frame of exposure, and the like, and one skilled in the art can appropriately select and adjust the incubation conditions. After incubation, the samples can be treated and processed in an appropriate manner so the sample can be analyzed.

In an embodiment, each of the compound probes of the library is injected into an animal (e.g., a mouse or tumor-bearing mouse). At different times after injection, each animal will be sacrificed, and organs, tissue, tumors, cells, and the like of the mouse can be processed in an appropriate manner so that they can be analyzed using MALDI-TOF-MS. In this way, the in vivo pharmacokinetic properties of each compound of the library can be determined.

In general, each of the samples or a portion thereof, either before and/or after treatment, is analyzed using a mass spectrometry system. In other words, the amount of each compound accumulated by each sample is quantified (e.g., relative to a standard) using a mass spectrometry system. In particular, the amount of each compound accumulated (e.g., present in the organelle, cell, tissue, tumor, and/or organ) by each sample and/or portions of each sample is quantified using a MALDI time-of-flight mass spectrometry system. Additional details regarding the mass spectrometry system are described below.

Screening Compound Probes

As mentioned above, after each compound probe is introduced to a sample and incubated, one or more compound probes that satisfy one or more selection criteria (e.g., uptake, concentration, location in a sample, biodistribution, combinations thereof, and the like) are selected as molecular imaging probes and can be further investigated (e.g., labeled with a radioisotope and analyzed). A mass spectrometry system is used to measure the amount of compound probe in the sample. The selection criteria can include, but are not limited to, the concentration of the compound probe detected relative to the standard and/or to one or more of the other compound probes in the sample, and the like. For example, the selection criteria can be based on the uptake of each compound probe into a cell, tissue, organ, or the like. One skilled in the art can determine the criteria, and the determination depends, at least in part, on the compound probe, the type of sample, the application, and the like.

In other words, embodiments of the present disclosure provide high throughput methods of screening (e.g., for probes that satisfy certain criteria) a library of compound probes to reduce the number of compound probes to be further analyzed (e.g., subject to more expensive testing), where the compound probes in the library are unlabeled but include an element that can be switched to its corresponding radioactive isotope for further analysis. As a result, time and expense can be saved because each of the compound probes in the library, which can include hundreds or thousands of compounds, do not have to be labeled to be screened using embodiments of the present disclosure.

The compound probes can include, but are not limited to, molecular imaging probes (initially having non-radioactive isotopes), and other imaging agents (e.g., PET agents, SPECT agents, MRI agents, CT agents, optical agents, and the like). Exemplary embodiments of non-radioactive elements and their counterparts include, but are not limited to, F-19 (F-18), C-12 (C-11), I-127 (I-125, I-124, I-131, I-123), Cl-36 (Cl-32, Cl-33, Cl-34), Br-80 (Br-74, Br-75, Br-76, Br-77, Br-78), Re-185/187 (Re-186, Re-188), Y-89 (Y-90, Y-86), Lu-177, and Sm-153. In particular, the non-radioactive elements and their counterparts include F-19 (F-18), Cl-36 (Cl-32, C-33, Cl-34), Br-80 (Br-74, Br-75, Br-76, Br-77, Br-78), and I-127 (I-125, I-124, I-131, I-123).

In one embodiment, the compound probes are mitochondria-targeting compound probes. In particular, the mitochondria-targeting compound probes include phosphonium cation compound probes. The library of compound probes can include, but is not limited to, (4-bromobutyl) triphenyl-phosphine bromide (BrTPP), butyltriphenylphosphonium chloride (BuTPP), (4-carboxybutyl)triphenyl-phosphonium bromide (CoTPP), methyltriphenyl phosphonium bromide (MeTPP), tetraphenylphosphonium bromide (TPP), triphenyl (2-pyridylmethyl)phosphonium chloride hydrochloride (PyTPP), tetrabutylphosphonium bromide (TBuP), Carbonylcyanide-m-chlorophenyl-hydrazone (CCCP), 4-Fluorophenyltriphenyl phosphonium (FTPP), tetra-4-fluorophenylphosphonium iodide (F4TPP), and 4-fluorobenzyl-triphenylphosphonium iodide (FBnTP). Additional details are described in Example 1 below.

In general, embodiments of the present disclosure provide for methods of constructing or preparing molecular imaging probes from a library of compound probes. The method includes constructing, selecting, and/or producing a library of compound probes as described above. The compound probes may include one or more non-radioisotopes of one or more elements (which can be subsequently exchanged for the radioisotope if additional experimentation is to be conducted). For example, in embodiments using prospective PET or SPECT agents, the compound probes contain the non-radioactive isotopes. If one or more compound probes meet certain criteria (e.g., show reasonable accumulation in cells or in vivo as determined by MALDI-MS) the non-radioactive isotopes can then be switched to their radioactive counterpart isotope to form the labeled compound probes for further analysis.

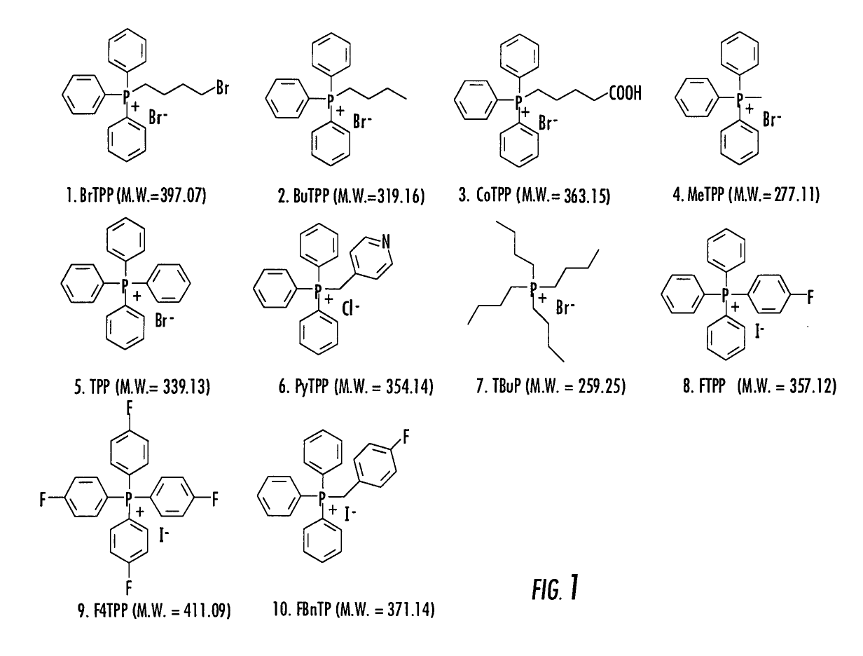

For example, in FIG. 1 (described in more detail below) compound 8 FTPP contains fluorine-19, which is non-radioactive. Since FTPP has good cell uptake, F-19 can then be switched to fluorine-18, which is radioactive, so it can be used as a PET or SPECT agent.

In another example, the labeling moiety may be a fluorophor, a MRI contrast agent, or a CT contrast agent that is already present in the compound probes. The in vitro or in vivo uptake can then be tested using MALDI-MS.

As mentioned above, the sample or a portion thereof, either before or after treatment, is analyzed using a mass spectrometry system. In other words, the amount of each compound accumulated by each sample is quantified (e.g., relative to a standard) using a mass spectrometry system. In particular, the amount of each compound accumulated by each sample is quantified using a MALDI time-of-flight mass spectrometry system as described in more detail above.

As described in more detail herein, the mass spectrometry system can be used, among others, to: quantify the cellular uptake of each compound probe into a cell, quantify the in tissue uptake of each compound probe into a tissue, determine the biodistribution of each compound probe in the tissue, quantify the in vivo organ uptake of each compound probe into an organ, and identify and/or qualify the compound probes associated with selected target molecules.

Methods of Screening Compound Probes

Embodiments of the present disclosure can be used to quantify the cellular uptake of each compound probe into a cell. In this regard, the library of compounds can be screened for compounds having a particular uptake into the cell (e.g., superior uptake over a certain time frame under certain conditions). The library of compounds can also be screened for compounds having a particular distribution within the cell and/or a particular uptake into cellular organelles (e.g., nucleus, mitochondria, etc.). The compounds having a particular uptake can be further analyzed. For example, one or more of the non-radioactive isotopes of the selected compound probes can be substituted with the radioactive isotopes for further testing.

It should be noted that the way in which the cell is treated before being introduced to the mass spectrometry system depends, at least in part, on the mass spectrometry system, the cell or cell culture, the compound probes, and the like. One skilled in the art can appropriately select and adjust the treatment conditions.

In another embodiment, the mass spectrometry system can be used to quantify the in-tissue uptake of each compound probe into a tissue. In this regard, the library of compounds can be screened for compounds having a particular in-tissue uptake (e.g., superior in-tissue uptake over a certain time frame under certain conditions). The compounds having a particular in-tissue uptake can be further analyzed. For example, one or more of the non-radioactive isotopes of the selected compound probes can be substituted with the radioactive isotopes for further testing.

It should be noted that the way in which the tissue is treated before being introduced to the mass spectrometry system depends, at least in part, on the mass spectrometry system, the tissue, the compound probes, and the like. One skilled in the art can appropriately select and adjust the treatment conditions.

In another embodiment, the mass spectrometry system can be used to determine the biodistribution of each compound probe in the tissue. For example, a MALDI time-of-flight mass spectrometry system can be used to sample portions of the tissue in a known grid-like manner. In this way, the MALDI time-of-flight mass spectrometry system can quantify the amount of compound probe at particular locations of the tissue, which can be subsequently evaluated. The way in which the tissue is treated before being introduced to the mass spectrometry system depends, at least in part, on the tissue, the compound probes, and the like. One skilled in the art can appropriately select and adjust the treatment conditions.

In another embodiment, the mass spectrometry system can be used to quantify the in vivo organ uptake of each compound probe into an organ. In this regard, the library of compounds can be screened for compounds having a particular in vivo organ uptake (e.g., superior in vivo organ uptake over a certain time frame under certain conditions). The compounds having a particular in vivo organ uptake can be further analyzed. For example, one or more of the non-radioactive isotopes of the selected compound probes can be substituted with the radioactive isotopes for further testing.

The way in which the organ is treated before being introduced to the mass spectrometry system depends, at least in part, on the mass spectrometry system, the organ, the compound probes, and the like. One skilled in the art can appropriately select and adjust the treatment conditions.

In another embodiment, the mass spectrometry system can be used to determine the specificity and/or affinity of each compound probe for a target molecule bound to an array or other substrate. In one embodiment, the substrate is a MALDI plate.

The term “array” encompasses the term “microarray” and refers to an ordered array presented for binding to the compound probes directly or indirectly. An “array,” includes any two-dimensional or substantially two-dimensional (as well as a three-dimensional) arrangement of addressable regions including target molecules or other probes. The target molecules may be adsorbed, physisorbed, chemisorbed, and/or covalently attached to the arrays. An array is “addressable” when it has multiple regions of different moieties (e.g., different target molecules) such that a region (i.e., a “feature” or “spot” of the array) at a particular predetermined location (i.e., an “address”) on the array will detect a particular compound probe or detect the interaction of a specific target molecule with a compound probe.

Mass Spectrometry

Mass spectrometry has gained prominence because of its ability to handle a wide variety of analytes with high sensitivity and rapid throughput. A variety of ion sources have been developed for use in mass spectrometry. Many of these ion sources include some type of mechanism that produces ions in accordance with an ionization process. One particular type of ionization process that is used is Matrix Assisted Laser Desorption Ionization (MALDI). MALDI is a technique used to produce ions for mass spectrometry. One benefit of MALDI is its ability to produce ions from a wide variety of analytes. Another benefit of MALDI is its ability to produce ions with reduced fragmentation, thus facilitating identification of analytes from which the ions are produced.

Typically, the MALDI (ion source) produces ions from a co-precipitate of an analyte and a matrix. The matrix can include organic molecules that exhibit a strong absorption of light at a particular wavelength or a particular range of wavelengths, such as in the ultraviolet range. For a conventional MALDI mass spectrometry system, an analyte and a matrix are dissolved in a solvent to form a solution, and the solution is then applied to or positioned on a sample support. As the solvent evaporates, the analyte and the matrix form a co-precipitate on the sample support. The co-precipitate is then irradiated with a short laser pulse that induces an accumulation of energy in the co-precipitate through electronic excitation or molecular vibration of the matrix. As the matrix dissipates the energy by desorption, the matrix carries the analyte into a gaseous phase. During this desorption process, ions are produced from the analyte by charge transfer between the matrix and the analyte.

It is also contemplated that the ion source can be implemented to produce ions using any other ionization process, such as vacuum MALDI or Atmospheric Pressure-Matrix Assisted Laser Desorption Ionization (“AP-MALDI”), Atmospheric Pressure Photo Ionization (“APPI”), and the like. It is also contemplated that the ion source can be implemented as a multi-mode ion source that produces ions using a combination of ionization processes.

The mass spectrometry system also includes a detector system, which is positioned downstream with respect to the ion source to receive ions. The detector system operates to detect ions as a function of mass to charge ratio. The detector system includes a mass analyzer, which operates to separate or select ions by mass-to-charge ratio. One embodiment of the mass analyzer includes a time-of-flight analyzer. However, it is contemplated that other types of mass analyzers can be used, such as ion trap devices, quadrupole mass spectrometers, magnetic sector spectrometers, and the like. The mass spectrometry system could also include other components known to one of skill in the art.

While embodiments of the present disclosure are described in connection with the following example and the corresponding text and figures, there is no intent to limit the disclosure to the embodiments in these descriptions. On the contrary, the intent is to cover all alternatives, modifications, and equivalents included within the spirit and scope of embodiments of the present disclosure.

### EXAMPLE

Using matrix assisted laser desorption/ionization time of flight mass spectrometry (MALDI-TOF-MS) to screen molecular imaging probe uptake in cell culture without radiolabeling is explored in this Example. In MALDI-TOF-MS, a matrix is introduced into sample preparation to enable soft and efficient ionization of different samples ranging from small molecules to peptides and proteins. Then time of flight (TOF) system is usually incorporated for coupling with matrix assisted laser desorption/ionization (MALDI) sources, rendering this technique the capacity to examine a very broad mass range. MALDI-TOF-MS is an analytic tool with advantages including high-speed analysis and sample throughput, high detection sensitivity and relatively low cost. This example describes the use of this technique for high-throughput screening and characterization of the mitochondrial targeting property of a library of phosphonium cations (FIG. 1) in order to facilitate the development of radiolabeled agents for imaging mitochondrial dysfunction.

Mitochondria are vital cellular organelles that play a central role in the energy metabolism of the cell. They are also involved in cell apoptosis and cardio-protection. Consequently, mitochondrial dysfunction contributes to a variety of human diseases such as cancer, diabetes, obesity, neurodegenerative disorders and ischemia-reperfusion injury. Lipophilic cations such as tetraphenylphosphonium (TPP) and the fluorescent dye rhodamine-123 (Rh123) have an affinity to and accumulate selectively in the mitochondrial matrix due to the combination of elevated plasma and mitochondrial membrane potentials (Δψm). The lipophilic cations [¹¹C] triphenylmethylphosphonium and [³H]TPP can function as molecular imaging probes for monitoring diseases that involve mitochondrial damage. Considering the rapid increase in research and application of this class of agent, a technique that can quickly identify and characterize additional useful lipophilic cations for mitochondrial imaging will definitely move the mitochondrial medicine field forward significantly. Furthermore, many of these delocalized organic cations display anti-neoplastic effects both in vitro and in vivo and have been actively explored as a drug delivery system that targets mitochondria. TPP analogs, such as cationic triarylalkyl-phosphonium salts, are particularly interesting because of their high synthetic accessibility for conjugation with a variety of biological motifs. Therefore, the MALDI-TOF-MS approach for fast screening phosphonium cations for cell targeting capability should help the development of not only molecular imaging probes but also drug carriers and anticancer agents.

In this Example, MALDI-TOF-MS was used to compare the amount of phosphonium cations accumulated in cultured glioma C6 cells and thus determine the ability of these cations to penetrate biological membranes. The use of MALDI-TOF-MS allowed the quick identification of candidate probes that are worthy of further development for in vivo imaging applications. These results demonstrate that MALDI-TOF-MS is a powerful analytical tool for rapid screening and characterization of phosphoium cations, as well as other molecules, as molecular imaging probes.

Materials and Methods

Materials: Alpha cyano-4-hydroxy-cinnamic acid (αCHCA), (4-bromobutyl) triphenyl-phosphine bromide (Compound #1, BrTPP), butyltriphenylphosphonium chloride (Compound #2, BuTPP), (4-carboxybutyl)triphenyl-phosphonium bromide (Compound #3, CoTPP), methyltriphenyl phosphonium bromide (Compound #4, MeTPP), tetraphenylphosphonium bromide (Compound #5, TPP), triphenyl(2-pyridylmethyl)phosphonium chloride hydrochloride (Compound #6, PyTPP), tetrabutylphosphonium bromide (Compound #7, TBuP), 4-fluorobenzyl-triphenylphosphonium iodide (Compound #10, FBnTP), Carbonylcyanide-m-chlorophenyl-hydrazone (CCCP), and valinomycin were purchased from Sigma-Aldrich Chemical Co., Fluka (St. Louis, Mo.) and Alfa Aesar (Ward Hill, Mass.). 4-Fluorophenyltriphenyl phosphonium (Compound #8, FTPP) was synthesized by the method reported previously (see, Label Compd Radiopharm, Vol. 48, Pages 131-137, which is incorporated by reference). Tetra-4-fluorophenylphosphonium iodide (Compound #9, F4TPP) was synthesized using a similar synthetic approach as for making FTPP. Stock solutions of the phosphonium cations for cell uptake studies were prepared by dissolving in 1M HEPES buffer and diluting to the desired concentration. ³H-TPP [1.11 TBq/mmol (30 Ci/mmol), 37 kBq/μL (1 μCi/μL)] was obtained from Moravek Biochemicals, Inc.

MALDI-TOF Mass Spectrometry: MALIDI-TOF-MS experiments were performed on a Perseptive Voyager-DE RP Biospectrometry instrument (Framingham, Mass.). On the MALDI-TOF-MS target plate, 1 μL each of sample solution and 1 μL of the matrix were mixed. Alpha-CHCA (prepared as 10 g/L in 33.3% AcCN: 33.3% EtOH: 33.3% H₂O: 0.1% TFA) was used as solid matrix. All samples were analyzed under the following conditions: reflectron positive ion mode with an accelerating voltage of 20 kV, a pulse delay time of 100 ns, a grid voltage of 74.5%, a guide wire voltage adjusted to 0.05%, laser intensity 2710, and 120 shots/spectrum. For each analysis, the laser spot randomly hit the sample to generate an averaged MALDI-TOF-MS spectrum. Three spectra were produced for each sample, and analyte to internal standard response ratios were averaged.

Cell Culture and Cell Uptake Studies of Phosphonium Cations: A rat glioma C6 cell line was grown in Dulbecco's modified Eagle medium (DMEM, Invitrogen Life Technologies, Carlsbad, Calif.), high glucose (containing KCl 5.3 mmol/L and NaCl 110.34 mmol/L) plus 10% fetal bovine serum (FBS), and 1% penicillin-streptomycin at 37° C. in 5% CO₂/95% air. After trypsinization, cells were washed twice with 0.01 M phosphate-buffered saline (PBS, pH=7.4) and suspended at 10×10⁶ cells /mL in low K⁺ HEPES buffer (135 mM NaCl/5 mM KCl/1.8 mM CaCl₂/0.8 mM MgSO₄/50 mM HEPES [4-(-2-hydroxyethyl)-1-piperazineethanesulfonic acid]/5.5 mM dextrose, pH 7.4). C6 cells (0.5×10⁶/50 μL) were incubated with 10 μL 100 μM stock phosphonium salts solution (5 μM final concentration) and 140 μL HEPES buffer from 10 to 90 min at room temperature. The cells were centrifuged (250×g) for three minutes and washed twice with cold PBS buffer at specified times. Cell pellets were then lysed with 150 μL cold spectral grade water, followed by freezing in dry ice. Right before the MALDI-TOF-MS analysis, the frozen cell solution was thawed and centrifuged at 12000×g for 5 min. An aliquot of 90 μL of cell lysate and 10 μL of 10 μM internal standard MeTPP were mixed together, and 1 μL of solution was subjected to MALDI-TOF-MS analysis. Samples collected from three independent cell lysates were prepared for each study and subjected for MALDI-TOF-MS analysis.

Calibration Curves: Phosphonium cations, BrTPP (#1), BuTPP (#2), CoTPP (#3), MeTPP (#4), TPP (#5), PyTPP (#6), TBuP (#7), FTPP (#8), F4TPP (#9), and FBnTP (#10) were dissolved in methanol. Then they were diluted with C6 cell lysate (0.5×10⁶ cells per 150 μL, these cells were not incubated with any phosphonium compound prior to lysis) to make various concentrations of compound and stored at 0° C. One micromolar MeTPP in 10 μL volume, which was used as internal standard, and 10 μL phosphonium cations with concentrations ranging from 0.1 μM-1.5 μM were mixed together thoroughly. Subsequently, 1 μL aliquot and 1 μL matrix αCHCA were deposited on the target plate and dried for MALDI-TOF-MS analysis. Triplicate samples were prepared and analyzed.

Cell Uptake of ³H-tetraphenylphosphonium (³H-TPP): C6 cell uptake of ³H-TPP was also performed for direct comparison with cell uptakes of TPP and FTPP determined through MALDI-TOF-MS. Briefly, 0.5×10⁶ cells were incubated with 5 μM ³H-TPP [³H-TPP was spiked with TPP to make a final concentration of 12.21 GBq/mmol (0.33 Ci/mmol)] from 10 to 90 min in a 200 μL HEPES buffer at room temperature. Cell lysate was prepared using the same procedure as described in the Cell Uptake Studies of Phosphonium Cations section, and the radioactivity of cell lysate was counted. Triplicate samples were obtained for all uptake studies. Data were expressed as the accumulation of the amount of probe in each cell or percent cell uptake (fmols/cell, % cell uptake). ³H analyses were performed with a Beckman LS-6500 liquid scintillation counter with Biosafe II scintillation fluid (Research Products International, Mount prospect, Ill.). The attenuation and quenching effects resulting from the cell lysate used in this experiment were determined to be negligible, and the disintegration per minute (dpm) was obtained by correcting for background activity and efficiency (69.3% for ³H).

Mitochondrial Membrane Potential (MMP) Dependent Uptake of FTPP, F4TPP and FBnTP: The extent of MMP and cell membrane potential-dependent cellular uptake of FTPP, F4TPP, and FBnTP was further assessed using the C6 cells treated with CCCP, valinomycin, and high K⁺ HEPES buffer (5 mM NaCl/135 mM KCl/1.8 mM CaCl₂/0.8 mM MgSO₄/50 mM HEPES/5.5 mM dextrose, pH 7.4). CCCP, a known protonphore that selectively abolishes the MMP (Steen H, Maring J G, Meijer D K F. Differential effects of metabolic inhibitors on cellular and mitochondrial uptake of organic cations in rat liver. Biochemical Pharmacology. 1993; 45:809-818, which is incorporated herein by reference), was dissolved in dimethyl sulfoxide (DMSO) and diluted to desired concentration with low K⁺ HEPES buffer. K⁺-ionophore valinomycin was dissolved in ethanol and also diluted with low K⁺ buffer. Final concentrations of ethanol and DMSO were below 0.1%. Varying concentrations of CCCP and valinomycin were added to the 0.5×10⁶ cells in low K⁺ HEPES buffer 30 min prior to the start of the experiment. After one hour of incubation with 5 μM FTPP, F4TPP, or FBnTP, the eppendorf vials were centrifuged, cell lysate were prepared as described above and the cell uptakes of FTPP, F4TPP, or FBnTP were determined using MALDI-TOF-MS. Five sets of 0.5×10⁶ cells were also suspended in high K⁺ HEPES buffer for 60 min, then exposed to 5 μM FTPP, F4TPP, or FBnTP for 10-90 min in high K⁺ HEPES buffer. Cell uptake of FTPP, F4TPP, and FBnTPP was measured by MALDI-TOF-MS. The viability and the integrity of the cells after treatment with inhibitors and high K⁺ buffer were examined using Trypan blue. It showed the cells were not affected by the inhibitors and high K⁺ buffer used within the time period when uptake of the phosphonium analogs was measured.

Statistical analysis: Statistical analysis was performed using the Student's t test for unpaired data. A 95% confidence level was chosen to determine the significance between two groups, with P<0.05 being considered significantly different. Linear regression analysis was performed to assess the linear relationship between the MALDI-TOF-MS ion-intensity ratios (phosphonium cations (PCs)/MeTPP) and the mole ratios (PCs/MeTPP), with 1.0 μM MeTPP as an internal standard. The lines are based on linear least-squares fitting of each of these sets of data points. The degree of correlation between them was quantified in terms of the square of Pearson product moment correlation coefficient (r²).

Results

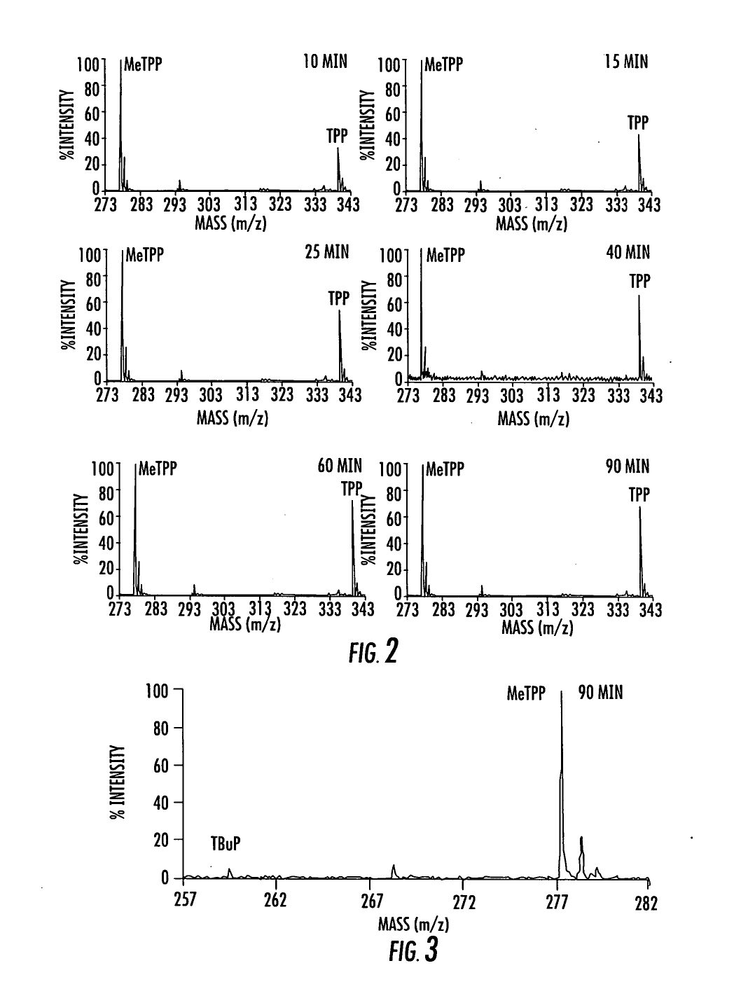

Quantitative Analysis using MALDI-TOF-MS: Alpha-CHCA is a very popular matrix and useful for analysis of low molecular weight (LMW) compounds. The preliminary results demonstrated, for all phosphonium cations shown in FIG. 1, that no fragment peaks and Na or K ion adducts were observed, and only the [M]⁺ ions showed good signal without interference with αCHCA matrix ion peaks. This made quantitative analysis readily possible and reproducible. Therefore the αCHCA matrix was used to study C6 cell uptake of different phosphonium cations. Furthermore, in order to maximize intra (sample-to-sample) and inter (point-to-point and shot-to-shot signal) experimental reproducibility, an internal standard is usually used for quantitative analysis in the low molecular weight (LMW) range. In this example, MeTPP was used as an internal standard for measurement of the amount of phosphonium cations presented in the cell using MALDI-TOF-MS due to its chemical similarity. To determine the cell uptake of MeTPP itself, TPP can be used as an internal standard. FIG. 2 shows the representative MALDI-TOF-MS spectra for the time course of TPP cell uptake. MeTPP and TPP can be easily assigned and labeled separately on the spectra. In FIG. 2, the peaks without labels were generated by the matrix. Ion intensity of internal standard, MeTPP, was set as 100. It can be seen from the spectra that the ion intensity of analyte, TPP, increases over time and reaches a maximum uptake at 60 min incubation time. The peak area of the [M]⁺ (MeTPP: m/z 277.1 or TPP: m/z 339.1) ion was then determined, and ion intensity ratio (TPP/MeTPP) was calculated. Using the calibration curve described below, the amount of TPP in the cells at each time point was quantified. A representative MALDI-TOF-MS spectrum of the cell uptake of TBuP at 90 min is also presented in FIG. 3 for comparison with FIG. 2. Although the TBuP peak ([M]⁺: m/z 259.2) is small, it is still quite easy to identify the peak and follow the change in peak intensity since the signal to noise ratio is at least over 5. The lower TBuP/MeTPP than TPP/MeTPP suggests lower cell uptake for TBuP than TPP.

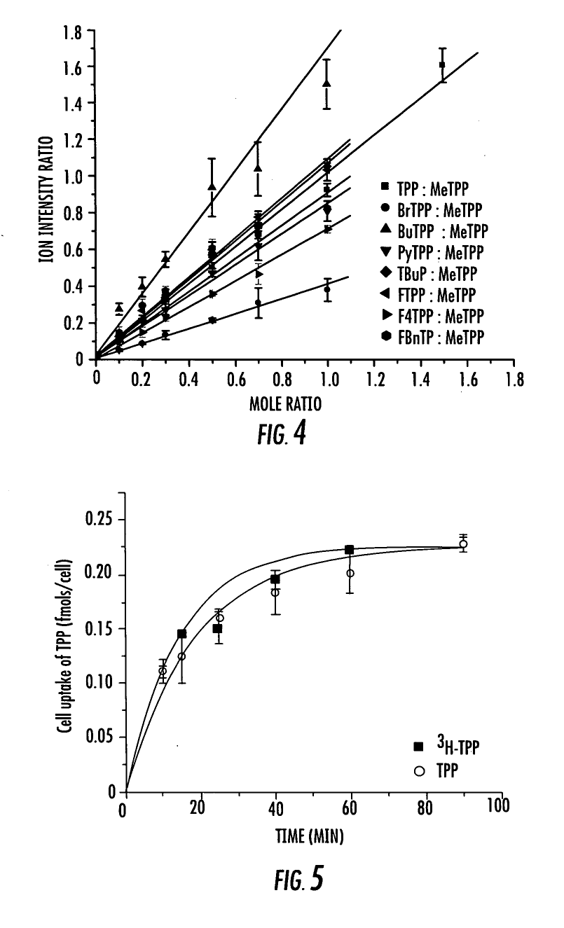

To obtain the calibration curves for quantitative analysis, a series of phosphonium cations containing 0.1, 0.2, 0.3, 0.5, 0.7, 1.0, and 1.5 pmol analytes in 1 μL solutions and 1.0 pmole internal standard (MeTPP) in 1 μL solutions were mixed together and subjected to MALDI-TOF-MS analysis. Triplicate samples were prepared, and three averaged spectra were collected for each sample. The I_{PCs/MeTPP} (PCs refers to phosphonium cations) was measured as the ratio of the [M]⁺ ion peak area. The plotted data are shown in FIG. 4. A very good correlation between MALDI-TOF-MS ion-intensity ratios [PCs/MeTPP] and the mole ratios (PCs/MeTPP) is observed. The linear correlation coefficients (r²) are all greater than 0.98. Furthermore, the slopes of the calibration curves are different for each cation, indicating different detection sensitivity for these cations.

Comparison of Cell Uptake of TPP Measured by MALDI-TOF-MS and Scintillation Counting: C6 cells at a concentration of 0.5×10⁶ were exposed to 5 μM TPP and ³H-TPP separately by incubating at 25° C. over a period of 90 min. The drug-influx kinetics were followed either by MALDI-TOF-MS for TPP or by scintillation counting for ³H-TPP. The time course of TPP uptake, as determined with the two techniques, exhibited an almost identical pattern and the same levels of uptake (p<0.05) (FIG. 5).

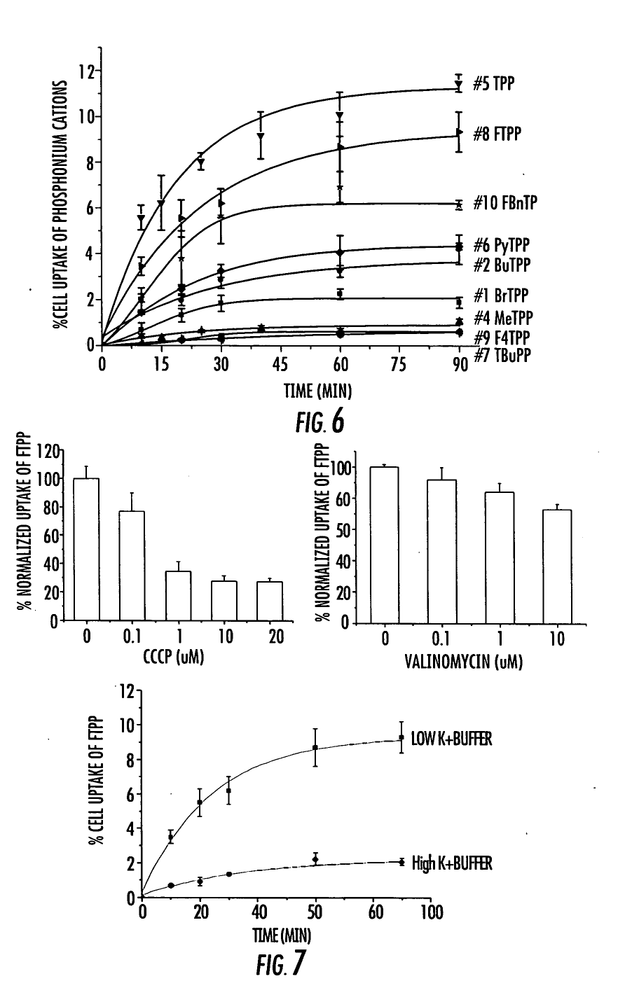

Cell Uptake of Phosphonium Cations: FIG. 6 shows the cell uptake of phosphonium cations (5 μM was used for incubation for all experiments) on C6 cells at 25° C. over a 90 min incubation period. The phosphonium cations exhibit very similar cell uptake kinetics. The cations rapidly increase in cells upon introduction into the buffer and reach steady state concentration at 60 min. The percentage of cell uptake for various phosphonium cations display different levels of steady-state, ranging from 0.6% to 11.4%. Interestingly, the molecular peak, fragments, and molecular adduct for the compound CoTPP could not be identified from MALDI-TOF-MS for the cell lysate resulting from incubation with CoTPP. This result can be explained by the fact that CoTPP is a neutral compound at physiological condition, and either it could not enter cells or the accumulation was lower than the detection sensitivity.

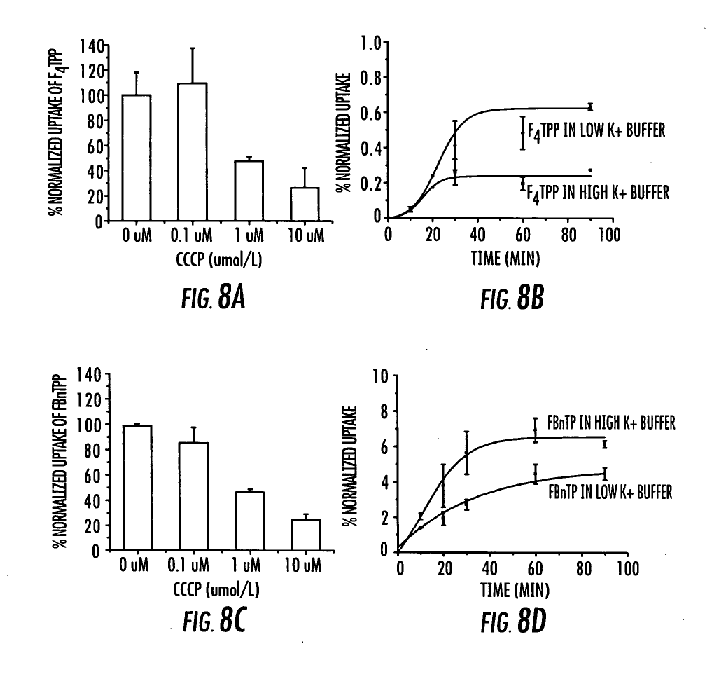

MMP Dependent Uptake of FTPP: In order to assess the effects of manipulating mitochondrial membrane potential on the cellular accumulation of FTPP, F4TPP, and FBnTP (determined by MALDI-TOF-MS), uptake studies were performed for C6 cells under 4 different concentrations of CCCP (0.1, 1, 10, 50 μM) and 3 different concentrations of valinomycin (0.1, 1, 10 μM) in low K⁺ HEPES buffer. For control experiments, in which the mitochondrial membrane potentials were unaltered, uptake was determined in a near physiological buffer (low K⁺ HEPES buffer) without adding any inhibitors. The results are depicted in FIGS. 7A and 7B, and FIGS. 8A and 8C. It can be seen that the uptake of FTPP in the C6 cells is significantly inhibited (p<0.02) by both CCCP or valinomycin at 1 μM concentration. The FTPP uptake is highly inhibited by 50 μM CCCP to about 27.3±2.3%, and to a lesser extent by 10 μM valinomycin to about 72.8±3.6% (FIG. 7). The uptake of F4TPP and FBnTP in C6 can also be inhibited by CCCP at 1 μM concentration.

The effects of depolarizing the plasma membrane potential only on the C6 cell uptake of the phosphonium cations were obtained by using a high K⁺ HEPES, as shown in FIG. 7C and FIGS. 8B and 8D. For C6 rat glioma cells, depolarization of the plasma membrane caused a 3.5-fold decrease in uptake of FTPP, a 3.0-fold decrease in uptake of F4TPP, and a 1.5-fold decrease in uptake of FBnTP, compared to their uptakes in the low K⁺ buffer at 90 min exposure time. Overall, these results clearly demonstrate that the uptake of FTPP, F4TPP and FBnTP is electrogenic and driven by the plasma and mitochondrial membrane potentials.

Discussion

One of the fruits of the Human Genome Project is that identification of new targets for diseases is being greatly accelerated, which urges scientists to develop new techniques for rapid screening of molecules that are able to interact with these targets. Mass spectrometry (MS) is one such technique that has attracted significant interest due to its great potential applications in high-throughput drug discovery and development. For instance, liquid chromatography combined with atmospheric pressure ionization (API) mass spectrometric detection (LC-MS/MS) has been actively investigated for the determination of drugs' pharmacokinetic (PK) properties and their metabolites. A novel method using liquid chromatography tandem mass spectrometry (LC/MS/MS) was developed to monitor the cellular uptake profile of a non-radioactive drug, Paclitaxel. Typically, incubation of radiolabeled compounds with cells in culture is employed for detecting the amounts of drug accumulated in the cells. However, to radiolabel a series of compounds can be very time consuming, expensive, and sometimes, a very challenging task for organic radiosynthesis. The bioanalytical method of the present disclosure avoids preparing radiolabeled version of the drug. The method is rapid, sensitive, and presents a unique advantage over traditional radioisotopic methods in that it can readily be employed on a range of analogs without any additional synthetic effort.

Currently, LC-MS/MS is the most popular MS technique for high-throughput assays. But the chromatography separation involved in this technique significantly extends the total analysis time. It takes at least several minutes to finish one run. Large amounts of solvent waste are also generated from the assay. Moreover, LC-MS method development and validation are very time-consuming and require technically skilled scientists. MALDI-TOF-MS is another very powerful analytical technique with intrinsic advantages including high speed of analysis (several seconds per sample) and high detection sensitivity (pmole range), making it a potential tool for high-throughput screening of drugs. MALDI-TOF-MS can be a powerful tool for quantitative analysis of low molecular weight compounds. For example, MALDI-TOF-MS has already been successfully used for quantitative analysis of an enzyme catalyzed reaction, protease activity, and cyclosporin A in biological samples. It was also reported as a potential method for detecting trace-level (in ppb range) drug residues in complex environmental samples. Therefore, in this Example, MALDI-TOF-MS was used for shortening the molecular imaging agent development timeline.

First, the same amount of non-radioactive TPP and ³H-TPP (1 nmol in 200 μL) were incubated with 0.5×10⁶ C6 cells. Cell uptake of ³H-TPP was easily determined for comparison with MALDI-TOF-MS analysis through routine scintillation counting of tritium labeled compound. As for quantitative analysis of the TPP accumulation in the cells using MALDI-TOF-MS technique, calibration curves for the internal standard, MeTPP, and TPP and other phosphonium cations were first established (FIG. 4). The results show high linear correlation and accuracy (B≈1 and r²>0.98 for all curves) during the mass range applied for the analysis in this research (0.1 pmol to 1.5 pmol for analytes and 1 pmol for internal standard). After incubation with cells at different time points, TPP from 3000 cell lysate and 1 pmol MeTPP as an internal standard together were sampled for analysis. MALDI-TOF-MS spectra clearly revealed that TPP could be easily detected from the cell lysate even at 10 mins post incubation (FIG. 2), and as low as 0.11 fmol TPP per cell (FIG. 5), as determined through the ion-intensity ratio and standard calibration curve. The time course of TPP influxes determined by MALDI-TOF-MS and scintillation counting shows no significant difference statistically (P>0.05 for all time points). Overall, these data validated MALDI-TOF-MS as an alternative approach for accurate measurement of the cell accumulation of phosphnoium cations.

The same procedure for MALDI-TOF-MS analysis was employed to determine cell accumulation of all the other phosphonium cations. Although the phosphonium analogs evaluated in this research display similar uptake kinetics, as presented in FIG. 5, the absolute value of their uptake is different. CoTPP, a zwitterion under physiological condition, either does not accumulate or accumulates minimally in the cells over a 90 minute incubation period, presumably due to its zero net charge. TBuPP is a phosphonium salt without any phenyl ring and shows very low cell accumulaiton (FIGS. 3 and 6). Compared to TPP, an appoximately 18-fold decreased cellular accumulation is observed for this compound (FIG. 6). Interestingly, cationic triarylalkylphosphonium salts (MeTPP, BrTPP, BuTPP, PyTPP, F4TPP, and FBnTP) all displayed much lower uptake than that of tetraphenylphosphonium analogs (TPP and FTPP). These results highlight the importance of fine tuning the structure of this class of compound for achieving the most efficient mitochondrial targeting. These results also indicate that, with careful selection, one or more of the phosphonium cations may also serve as a carrier for drug delivery for non-imaging applications.

As shown in FIG. 6, the percent cell uptake for MeTPP, FTPP and TPP is 1.06, 9.33 and 11.44, respectively, when they reach a plateau at ˜90 minute after incubation. This information is of particular value for developing radiolabeled compounds as molecular probes for disease imaging. We have demonstrated that ³H-TPP is specifically accumulated in tumors but has minimal accumulation in inflammatory sites because of its mitochondrial targeting property. Therefore, TPP labeling with F-18 may generate a novel molecular probe for mitochondria dysfunction imaging in vivo using positron emission tomography (PET). The results shown in FIG. 6 demonstrated that non-radioactive FTPP is still able to sustain over 80% of the cell uptake of that of TPP. Moreover, the plasma membrane and mitochondrial membrane potential of C6 cells were modulated either by using high K⁺ HEPES buffer or inhibitors such as CCCP and valinomycin. Cell uptake profiles of FTPP using these membrane potential modulated cells verified that FTPP still preserves the membrane potential dependence property of TPP in cell culture (FIG. 7A-C), even though a fluorine was incorporated into the parent molecule. In another report, using dynamic PET studies, ¹¹C-MeTPP exhibited enhanced uptake and prolonged retention in canine brain glioma. Considering FTPP has 8 times higher accumulation than MeTPP in cell culture (FIG. 6) and the longer half life of F-18, further studies to develop F-18 labeled TPP as a mitochondrial targeting PET agent are well justified after study of uptake of this probe by additional cell lines. Furthermore, FTPP exhibited higher C6 cell uptake than both F4TPP and FBnTP, suggesting FTPP as a lead compound in the library we constructed for further development.

The present evaluation of the time-course cell uptake of a library of compounds using MALDI-MS-TOF demonstrates that MALDI-MS-TOF analysis helps to identify and propose new imaging probes for further development. The voltage-sensitive nature of the lead compound FTPP was also proven using MALDI-MS-TOF.

In routine radioactive cell uptake assays, the amount of tracer employed for the assay is very dependent on the specific activity of the radiolabeled agent. It can range from pM to nM range. For instance, when 370 Bq (0.01 μCi) ³H-TPP (1.11 TBq/mmol (30 Ci/mmol)) is incubated with cells in 100 μl buffer, the concentration of ³H-TPP corresponds to 3.3 nM. In this experiment, phosphonium cations and ³H-TPP at 5 μM concentration were used for incubation with cells. Although the concentration of cold phosphonium cations used for MALDI-TOF-MS cell assay is over thousand times higher than radiotracer routinely used in radioactive cell uptake assays, the procedure described in this paper can be fully optimized to scale down the initial incubation concentration of cold compounds. For instance, 0.5×10⁶ C6 cells were used for incubation with 5 μM phosphonium cations, and the cells were then lysed in 150 μl water. Only a very small fraction (1 μl, 0.67%) of the cell lysate was subjected to MALDI-TOF-MS analysis. Obviously, less volume of water can be used for cell lysis and/or the cell lysate can be condensed through lyophilization and re-dissolving. Moreover, instead of only using 0.5×10⁶ cells, a larger number of cells, for example, 5×10⁶ cells can be applied for incubation. Through a combination of the above two approaches it is expected that considerable improvements can be achieved in the sensitivity of MALDI-TOF-MS for quantification of cell uptake of molecular probes.

Imaging abnormalities of small animals or patients at the molecular level with PET technology has shown significant potential and has attracted significant attention in biomedical research. Increasing numbers of radiopharmaceuticals are in high demand for probing cellular or molecular targets. However, to develop and characterize radiolabeled agents in a rapid and systematic fashion still presents a very formidable task to radiochemists. Radio-LC-MS was utilized and proven to be a quick and accurate analytical tool for determining the chemical composition of ^{99m}Tc radiopharmaceuticals at the tracer level by Liu et. al (Liu S, Ziegler M C, Edwards D S. Radio-LC-MS for the Characterization of ^{99m}Tc-Labeled Bioconjugates. Bioconjugates. Bioconjugate Chem. 2000; 11:113-117, which is incorporated herein by reference). This technique can help people to quickly identify the right radiolabeled species during the radiosynthesis step; thus, it has the potential to speed up the development of ⁹⁹mTc labeled agents. The research presented here demonstrates a new strategy which may be used for high throughput screening of PET and Single Photon Emission Computerized Tommgraphy (SPECT) agents for much broader applications to molecular imaging.

### CONCLUSION

In conclusion, MALDI-TOF-MS is successfully utilized for high throughput screening and characterization of a library of phosphonium cations as mitochondrial targeting agents. Sub-picomole levels of phosphonium cations accumulated in the cells are easily identified by the procedures described in this article. The high specificity, sensitivity and high speed of MALDI-TOF-MS render it a powerful tool for molecular probe and drug development. The cell uptake data for non-radioactive FTPP warrants the further synthesis and evaluation of ¹⁸FTPP as a molecular probe for imaging mitochondrial dysfunction.

It should be emphasized that the above-described embodiments of the present disclosure are merely possible examples of implementations and/or merely set forth for a clear understanding of the principles of the disclosure. Many variations and modifications may be made to the above-described embodiment(s) of the disclosure without departing substantially from the principles of the disclosure. All such modifications and variations are intended to be included herein within the scope of the present disclosure and protected by the following claims.

We claim:

1. A method, comprising:
providing a library of unlabeled compound probes, wherein each compound probe contains a first element, wherein the first element has at least one corresponding radioisotope;
introducing each compound probe to a sample;
incubating each compound probe with the sample;
quantifying the amount of each compound accumulated by each sample using a mass spectrometry system;
selecting one or more compound probes based on criteria; and
labeling each of the selected compound probes by replacing the first element with one of the corresponding radioisotopes.

2. The method of claim 1, wherein the mass spectrometry system is a matrix assisted laser desorption/ionization (MALDI) time-of-flight mass spectrometry system.

3. The method of claim 1, wherein the sample is selected from: a cell, a tissue, a tumor, an organelle, an organ, and combinations thereof.

4. The method of claim 1, further comprising: quantifying the cellular uptake of each compound probe into a cell.

5. The method of claim 1, further comprising: quantifying the in vivo organ uptake of each compound probe into an organ.

6. The method of claim 1, further comprising: quantifying the in tissue uptake of each compound probe into a tissue.

7. The method of claim 6, further comprising: determining the biodistribution of each compound probe in the tissue.

8. The method of claim 7, further comprising: analyzing the tissue in sections to determine the biodistribution using the MALDI time-of-flight mass spectrometry system.

9. The method of claim 1, wherein the first element and the corresponding radioisotope are selected from the following: the first element is F-19 and the radioisotope is F-18; the first element is Cl-36 and the radioisotope is selected from Cl-32, Cl-33, and Cl-34; the first element is Br-80 and the radioisotope is selected from Br-74, Br-75, Br-76, Br-77, and Br-78; and the first element is I-127 and the radioisotope is selected from I-125, I-124, I-131, and I-123.

10. The method of claim 9, wherein the mass spectrometry system is a matrix assisted laser desorption/ionization (MALDI) time-of-flight mass spectrometry system.

11. A high throughput method for identifying molecular imaging probes in a library, comprising:
providing a library of unlabeled compound probes, wherein each compound probe contains a first element, wherein the first element has at least one corresponding radioisotope;
introducing each compound probe to a sample;
incubating each compound probe with the sample;
quantifying the amount of each compound accumulated by each sample using matrix assisted laser desorption/ionization (MALDI) time-of-flight mass spectrometry system; and
selecting one or more compound probes based on criteria.

12. A high throughput method for identifying molecular imaging probes in a library, comprising:
providing a library of compound probes, wherein each compound probe contains a label selected from: a fluorophor, MRI contrast agent, and CT contrast agent;
introducing each compound probe to a sample;
incubating each compound probe with the sample;
quantifying the amount of each compound accumulated by each sample using a mass spectrometry system;
selecting one or more compound probes based on criteria.

13. The method of claim 12, wherein the sample is selected from: a cell, a tissue, a tumor, an organelle, an organ, and combinations thereof.

14. The method of claim 12, wherein the mass spectrometry system is a matrix assisted laser desorption/ionization (MALDI) time-of-flight mass spectrometry system.

15. The method of claim 12, further comprising: quantifying the cellular uptake of each compound probe into a cell.

16. The method of claim 12, further comprising: quantifying the in vivo organ uptake of each compound probe into an organ.

17. The method of claim 12, further comprising: quantifying the in tissue uptake of each compound probe into a tissue.

18. The method of claim 17, further comprising: determining the biodistribution of each compound probe in the tissue.

19. The method of claim 18, further comprising: analyzing the tissue in sections to determine the biodistribution using the MALDI time-of-flight mass spectrometry system.
